(19) United States
(12) Patent Application Publication
(10) Pub. No.: US 2006/0105029 A1
(43) Pub. Date: May 18, 2006

(54) Instant patch for dermal drug delivery

(76) Inventors: Jie Zhang, Salt Lake City, UT (US); Kevin S. Warner, West Jordan, UT (US)

Correspondence Address: THORPE NORTH & WESTERN, LLP., 8180 SOUTH 700 EAST, SUITE 200, SANDY, UT, 84070, US

(21) Appl. No.: 11/271,114

(22) Filed: Nov. 10, 2005

Related U.S. Application Data
(60) Provisional application No. 60/627,555, filed on Nov. 12, 2004.

(51) Int. Cl. A61F 13/02; A61L 15/16

(52) U.S. Cl. 424/448

(57) ABSTRACT

The present invention is drawn toward systems, devices, and methods of dermal drug delivery. The system can comprise a gel-triggering agent and a drug-containing composition including a drug and a gelling agent, wherein the drug-containing composition forms a soft, coherent solid when contacted with the gel-triggering agent. A cavity patch is also included having an open cavity configured to be closed at least in part by a skin surface. The open cavity can be further configured to facilitate contact between the skin surface and the gel. The gel-triggering agent and the drug-containing composition can be positioned in the system such that they are kept isolated from one another until immediately before or during use.

The present application claims the benefit of U.S. Provisional Application No. 60/627,555, filed on Nov. 12, 2004, which is incorporated herein by reference in its entirety.

### FIELD OF THE INVENTION

The present invention relates to systems, devices, and methods for dermal drug delivery. More particularly, it concerns economical, convenient, and practical means for dermally delivering drugs.

### BACKGROUND OF THE INVENTION

Dermal patches are widely used as effective means of treating ailments and disease by delivering drugs and other active agents to the skin, regional tissues, and systemic circulation. Dermal patches are increasingly used because they have several advantages over semisolid formulations such as gels or creams. Some of the advantages of dermal patches include: being able to deliver drug to a well defined fixed area, protection of the drug formulation from the surrounding environment, and the ability to easily remove the patch after the intended application period is over. Although dermal patches have numerous advantages, the costs associated with the development, testing, and manufacture of dermal patches is generally significantly higher then those associated with traditional semisolid formulations. Because of the high costs associated with dermal patches, gels and creams continue to be the economically preferred choice for the treatment of many ailments and diseases, even though the use of dermal patches provides significant advantages to subjects.

### SUMMARY OF THE INVENTION

It has been recognized that it would be advantageous to provide a dermal drug delivery system that offers the advantages of traditional dermal patches and yet is economical, flexible in manner of use, and practical for many applications.

One embodiment of the present invention includes a system for dermal delivery of a drug. The system includes a gel-triggering agent, a drug-containing composition including a drug and a gelling agent. The drug-containing composition forms a soft, coherent solid when it is contacted with the gel-triggering agent. The system further includes a cavity patch having an open cavity configured to be closed at least in part by a skin surface. The open cavity is further configured to facilitate contact between the skin surface and the soft, coherent solid. The gel-triggering agent and the drug-containing composition are positioned in the system such that they are kept isolated from one another until immediately before or during use.

Another embodiment of the present invention provides for a method for dermal delivery of a drug. The method includes affixing a cavity patch to a skin surface, the cavity patch having an open cavity configured to be closed at least in part by the skin surface. The method further includes the step of contacting a drug-containing composition comprising a drug and a gelling agent with the gel-triggering agent such that a soft, coherent solid is formed within the cavity patch and then closing the open cavity.

Yet another embodiment of the present invention provides for a system for dermal delivery of a drug. The system includes a cavity patch having an open cavity configured to be closed at least in part by a skin surface, a gel-triggering agent disposed within the open cavity, and a drug-containing composition including a drug and a gelling agent. The drug-containing composition forms a soft, coherent solid when contacted with the gel-triggering agent. The gel-triggering agent and the drug-containing composition are positioned in the system such that they are kept isolated from one another until immediately before or during use.

In another embodiment, a device for facilitating dermal delivery of a drug can comprise a cavity patch having an open cavity which, when closed, is defined by one or more side wall, an impermeable top cover, and a skin surface; and a material within the cavity having a gel-triggering agent impregnated within or coated on the material. The device can be configured to be devoid of drug until immediately prior to or during use.

Additional features and advantages of the invention will be apparent from the detailed description, which illustrates, by way of example, features of the invention.

### BRIEF DESCRIPTION OF THE DRAWINGS

FIG. 1 is a schematic perspective view of a cavity patch in accordance with embodiments of the present invention;

FIG. 2 is a schematic perspective view of an alternative cavity patch in accordance with embodiments of the present invention; and

FIG. 3 is a schematic cross-sectional view of a cavity patch applied to a skin surface in accordance with one embodiment of the present invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and, “the” include plural referents unless the context clearly dictates otherwise. Thus, for example, reference to a drug-containing composition including “a drug” includes one or more drug components and reference to “the gelling agent” includes reference to one or more gelling agents.

As used herein, “subject” refers to a mammal that may benefit from the administration of the systems or methods of this invention. Examples of subjects include humans, and may also include other animals such as horses, pigs, cattle, dogs, cats, rabbits, and aquatic mammals.

As used herein, the terms “formulation” and “composition” are used interchangeably and refer to a mixture of two or more compounds, elements, or molecules.

As used herein, the term “gelling agent” refers to a compound or compounds which, when brought into contact with a gel-triggering agent, solidify into a soft solid composition. Gelling agents are generally well known in the art as are the corresponding gel-triggering agents which cause the gelling agents to solidify.

As used herein, the term “gel-triggering agent” refers to a compound or compounds which when brought into contact with a gelling agent cause the gelling agent to form a soft solid composition. “Boric acid” or a “salt of boric acid” is exemplary of compounds that when placed into contact with a gelling agent causes the gelling agent to form a soft coherent solid.

When referring to a gel-triggering agent that is “specific for gelling” a composition containing polyvinyl alcohol, this includes any gel-triggering agent that causes a drug-containing composition that includes polyvinyl alcohol to form a soft, coherent solid upon interaction therewith. Specific non-limiting examples include boric acid, a salt of boric acid, or a borate such as sodium, lithium, or potassium borate.

The terms “gelling agent” and “gel-triggering agent” are relative terms to one another. If two compounds form a gel when contacted, then one can be considered to be a gelling agent and the other a gel-triggering agent, or vice versa.

The term “viscosity modifying agent” refers to substances that can increase the viscosity of the formulation and compositions of the present invention. Non-limiting examples of modifying agents include polyvinyl alcohol, ethyl cellulose, a carbomer, hydroxy propyl cellulose, a methacrylic polymer and a methacrylate polymer.

The term “coherent” refers to solids which are formed that remain substantially intact with minimal or no ripping when gently removed from a skin surface, e.g., a composition that is peelable.

As used herein, the term “open cavity” or “drug reservoir” refers to the interior portion of the cavity patch which is capable of containing the drug-containing composition and the gel-triggering agent in accordance with embodiments of the present invention. The open cavity provides a retention form for the drug-containing composition and gel-triggering agent, preventing the composition from spilling, leaking, or running prior to the completion of the gelling of the drug-containing composition. The open cavity is capable of being closed at least in part by application to the skin of a subject. Generally the open cavity is defined by one or more patch wall(s) and an impermeable cover and can take on a variety of shapes and sizes. The patch wall(s) and impermeable cover can be integrated or modular. One preferred shape for the open cavity is a ring shape with an impermeable cover over one of the otherwise open ends.

As used herein, the term “skin contact region” refers to the area of the opening of the open cavity which is to be closed by skin, or in other words, the skin contact region defines the amount of skin surface which comes into contact with the drug-containing formulation contained in the open cavity. The skin contact region is limited by the walls of the cavity patch.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity, and thus, should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

The present invention is drawn to methods, devices, systems for dermal delivery of a drug. In one embodiment, the system includes a gel-triggering agent, a drug-containing composition including a drug and a gelling agent. The drug-containing composition forms a soft, coherent solid when it is contacted with the gel-triggering agent. The system further includes a cavity patch having an open cavity configured to be closed at least in part by a skin surface. The open cavity is further configured to facilitate contact between the skin surface and the soft, coherent solid. The gel-triggering agent and the drug-containing composition are positioned in the system such that they are kept isolated from one another until immediately before or during use.

Another embodiment of the present invention provides for a method for dermal delivery of a drug. The method includes affixing a cavity patch to a skin surface, the cavity patch having an open cavity configured to be closed at least in part by the skin surface. The method further includes the step of contacting a drug-containing composition comprising a drug and a gelling agent with the gel-triggering agent such that a soft, coherent solid is formed within the cavity patch and then closing the open cavity.

Yet another embodiment of the present invention provides for a system for dermal delivery of a drug. The system includes a cavity patch having an open cavity configured to be closed at least in part by a skin surface, a gel-triggering agent disposed within the open cavity, and a drug-containing composition including a drug and a gelling agent. The drug-containing composition forms a soft, coherent solid when contacted with the gel-triggering agent. The gel-triggering agent and the drug-containing composition are positioned in the system such that they are kept isolated from one another until immediately before or during use.

In another embodiment, a device for facilitating dermal delivery of a drug can comprise a cavity patch having an open cavity which, when closed, is defined by one or more side wall, an impermeable top cover, and a skin surface; and a material within the cavity having a gel-triggering agent impregnated within or coated on the material. The device can be configured to be devoid of drug until immediately prior to or during use.

In each of these embodiments, the drug-containing composition is preferably stored in a container capable of dispensing the composition into the cavity patch, optionally with a metered dose dispenser. The metered dose dispenser allows the user to dispense a pre-determined volume of the composition.

The configuration of the cavity patch for use in the dermal drug delivery systems and methods typically includes several advantageous features. For example, the system and methods 1) can allow for direct contact between the skin or mucosa by the drug-containing composition; 2) the cavity patch can retain the drug-containing composition solution (in pre- and post-gelled state) within a defined open cavity; 3) the cavity patch can remain affixed to the skin or mucosa for desired application time; and/or 4) when the patch is removed, no residue from the drug-containing composition is left behind. The desired application time varies depending on the desired result and the drug being administered. The desired application time can be as short as 0.25 hour and as long as 7 days, for example.

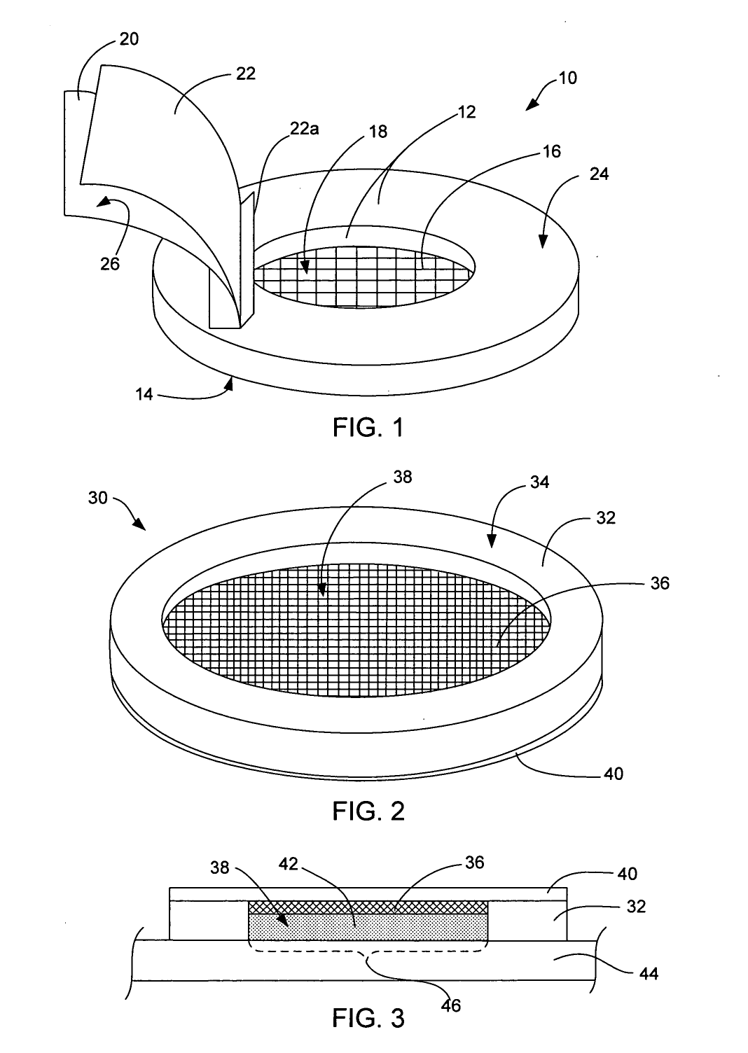

The cavity patch of the present invention has multiple effective configurations. In one configuration, the cavity patch can be top loading, as shown from a top perspective view at 10 in FIG. 1. In this embodiment, the cavity patch includes a wall 12 which is in the shape of a ring. The material of the wall can be an impermeable material, which is preferably a soft, flexible material, e.g., a closed-cell foam tape or other foam similar material. An adhesive 14 is typically coated on the wall to effectuate adhesion between the ring structure of the wall and a skin surface (not shown). In other words, the adhesive coated bottom wall is placed in direct contact with skin when the patch is in use. The cavity patch, as shown in FIG. 1, further includes an open cavity 18 (also referred to as the drug reservoir) being open on both ends like a donut to facilitate loading of a drug-containing composition into the cavity patch as well as facilitate contact between the drug-containing composition and the skin surface. One of the openings (on the bottom of the cavity patch) is substantially coextensive with the adhesive coated portion of the wall and is configured to be closed by application to the skin. The other opening (on the top of the cavity patch) is opposite the adhesive coated portion and is substantially coextensive with a top portion 24 of the wall. This opening is configured to be closed by an impermeable cover 20, preferably after application of the patch to skin and after the drug-containing composition is placed into the open cavity. The impermeable cover can include an adhesive substrate 26, and can have thermo-insulation properties in one embodiment. A release liner 22, with a leading edge 22a for gripping, is present that can be removed in order to protect the adhesive substrate until just prior to use. When the release liner is removed, the adhesive substrate of the impermeable cover can be used to close the open cavity at the top portion of the cavity patch.

The open cavity 18 is further configured to contain a gel-triggering agent. In the embodiment shown in FIG. 1, an absorbent mesh 16 impregnated or coated with the gel-triggering agent. The mesh material is used so that it does not close the open cavity, as the very nature of a mesh is that it has open areas between fibers or other material used to form the mesh. The gel-triggering agent is present which is capable of triggering a transition of the drug-containing composition from a more liquid or runny state to a more solidified coherent state or soft solid gel state. The transition from the liquid state to the soft, coherent solid gel state provides the advantage that when the patch is removed, the composition leaves little to preferably no residue on the skin.

To use the cavity patch shown in FIG. 1, the user adheres the cavity patch 10 to a skin surface with the adhesive 14 contacting the skin. The subject then places the drug-containing composition into the open cavity 18, and then closes or seals the open cavity with the impermeable adhesive cover 20 from which the release liner 22 has been removed. The gel-triggering agent on or impregnated within the mesh 16 diffuses into the drug-containing composition and causes the composition as a whole to gel into a soft, coherent solid over a period of time, thus forming a solidified patch that can be removed after desired application without leaving residue from the drug-containing composition on the skin. In accordance with embodiments of the present invention, the gel-triggering agent and the drug-containing composition are positioned in the system in such a way that they are kept isolated from one another until immediately before or during use of the cavity patch.

In an alternative configuration, the cavity patch 30 can be bottom loading, as shown from a bottom perspective view at in FIG. 2. A bottom portion of the ring shaped patch wall 32 is coated with an adhesive 34. The cavity patch has an open cavity 38 which is configured to be closed by application to a skin surface (not shown). Unlike the top loading cavity patch of FIG. 1, the bottom loading cavity patch of this embodiment has only one opening, the opening being coextensive with the bottom portion of the patch wall and the adhesive. The open cavity is defined by the patch wall and an impermeable cover 40 which is substantially coextensive with a top portion of the patch wall. Inside of the open cavity is a mesh or non-woven material 36 which is coated or impregnated with a gel-triggering agent, though any other material sufficient for carrying the gel-triggering agent can be used. In this embodiment, the mesh can be attached to the impermeable cover, or can be suspended at any location within the open cavity.

In accordance with FIG. 2, to use the device, the user places the drug-containing composition into the open cavity 38 of the cavity patch 30 with the bottom (adhesive side) up. When the drug-containing composition is loaded in the open cavity, the patch is placed onto the skin so that the adhesive 34 becomes affixed to the skin. The drug-containing composition is at least substantially sealed in the now closed open cavity by the impermeable cover 40, the patch wall 32 and the skin surface. Again, a mesh or non-woven material 36 (though any other material sufficient for carrying the gel-triggering agent can be used) is present. The gel-triggering agent in the mesh diffuses into the drug-containing composition and gels it into a soft solid after a certain amount of time, thus forming a solidified patch that can be removed after the desired application without leaving residue from the drug-containing composition on the skin. As mentioned above with respect to FIG. 1, in this embodiment as well, the gel-triggering agent and the drug-containing composition are positioned in the system in such a way that they can be kept isolated from one another until immediately before or during use of the cavity patch. When the drug administration period is complete, the patch can be removed from the skin and leave no substantial gel residue, if any, on the skin.

Turning to FIG. 3, a cross-sectional view of the cavity patch 30 of FIG. 2 is shown applied to a skin site 44. As mentioned above, this embodiment includes an impermeable cover 40, a patch wall 32, an open cavity 38 (which is shown as closed by the skin site), and a mesh or non-woven material 36 carrying a gel-triggering agent. However, in the embodiment shown, the drug containing composition 42 is loaded in the cavity (for interaction with the skin and the gel-triggering agent). The skin site includes a skin contact region 46 where the drug-containing composition contacts the skin surface. It should be noted that in the top loading system shown in FIG. 1, the skin contact region is found between the fibers of the mesh.

In each of these and other related embodiments, the skin contact region, which is one of the factors that dictates the amount of drug absorbed, is well defined by the area of the opening of the open cavity that is closed by the skin. This is an advantage over traditional semi-solid formulations such as ointments and creams, especially for drugs where precise dosing is beneficial or necessary. In terms of skin contact regions, the system of the current invention offers flexibility that traditional patches may not, namely a changeable skin contact region and easily modifiable dosage areas. If several different sizes are desired, patches with different sizes have to be manufactured and often individually packaged. In a typical GMP manufacturing process, each size usually has to be separately tested, released, and individually packaged, which can be very expensive. For example, nicotine patches for smoking cessation often come in three sizes. The largest size is used in the first phase of the treatment. As the nicotine craving decreases as the treatment progresses, smaller patches are used. Another example is the Duragesic® fentanyl patch which comes in 4 different sizes (10, 20, 30 and 40 cm²) to suit the pain control needs of different patients and different phases of the pain-causing diseases (e.g., cancer). In comparison, because open cavity patches of the present invention contain no active drug, the different size cavity patches of the current invention likely do not need to be separately tested and released, and do not need to be individually packaged. This can mean significant cost savings. A patient can merely select an appropriately sized patch, preferably under the supervision of a medical practitioner, and add an appropriate drug-containing composition to the cavity patch. In this respect, this type of system can be made to be modular or flexible in treatment type, drug dosage, etc.

As mentioned above, the skin contact region can be varied according to the needs of the desired application. For example, for treating a wart, an area of 0.1 to 1 cm² may be appropriate. For treating psoriasis or skin cancer, the area typically is significantly larger. Depending on the application and/or treatment regimen, the skin contact region can have an area of from 0.1 and 20 cm², and preferably from 0.2 to 10 cm². The open cavity can have a thickness or depth approximately equivalent to the thickness or depth of the cavity patch. The thickness (depth) of the open cavity can also be relevant to the performance of the system. If the open cavity is too shallow, the volume of the drug-containing composition that can be accommodated by the open cavity might be insufficient to ensure complete skin coverage with the drug-containing composition. If the depth of the open cavity is too great, it may take too long of a time for the gel-triggering agent to diffuse throughout the drug-containing composition to gel substantially the entire composition into a soft solid. In one embodiment, the depth of the open cavity can be from 0.1 mm to 5 mm, and more preferably between 0.2 mm to 3 mm.

As discussed in accordance with the FIGS., the open cavity includes a gel-triggering agent and the drug-containing composition includes a gelling agent capable of gelling the drug-containing composition into a soft solid state upon contact with said gel-triggering agent. Though the FIGS. show embodiments where the gel-triggering agent is coated or impregnated on a mesh structure, this is not required. For example, the gel-triggering agent can be coated on the (inner) walls of the open cavity patch, on the impermeable backing, or otherwise disposed within the cavity. Alternatively, the gel-triggering agent can be included in a dispensing device, e.g., a tube, to be co-dispensed into the open cavity with the drug-containing composition. In any of these or other embodiments, this design provides the convenience of placing a less-than-solid formulation into the open cavity and subsequently removing the used patch after use without leaving drug formulation residue on the skin.

Any pair of gelling agent(s) and gel-triggering agent(s) can be used in accordance with embodiments of the present invention, provided the agents do not substantially interfere with the functionality of the system. For example, agents that interfere with the desired functionality of the delivered drug, or which cause sensitivity or irritation of the skin may be less preferred, but are still included to the extent that they are functional. A good gel-triggering agent and gelling agent pair is boric acid (or a salt of boric acid or a borate) and polyvinyl alcohol.

To provide a more specific embodiment of an exemplary system, the open cavity can have a depth of 0.2 mm and a 5 cm² area, 20 mg of sodium borate (impregnated in a mesh which is disposed within the open cavity of the cavity patch, coated on the cavity patch within the open cavity, or on the adhesive coated substrate of the impermeable cover) can be present, and the drug-containing composition can include a drug and an appropriate water-based viscous fluid containing 15% polyvinyl alcohol. With this system, the drug-containing composition can be converted into a soft, coherent solid gel within about 30 minutes after being placed into the open cavity. The amount of boric acid or salt of boric acid which is impregnated into a mesh absorbent material can vary. In one embodiment, boric acid or the salt of boric acid can be impregnated into an absorbent material at a quantity from 1 to 20 mg/cm². In another embodiment, the amount of boric acid or the salt of boric acid impregnated into an absorbent material and disposed within the open cavity of the cavity patch can be from 4 to 8 mg/cm².

The impermeable cover can be made from materials with thermo-insulating properties such as closed-cell foam tape. Thermo-insulating materials can be advantageous if the drug in the drug-containing composition can better permeate the skin at elevated skin temperatures. For example, a patch in accordance with embodiments of the present invention with a thermo-insulating cover may be more effective in treating warts as it is believed that increased local skin temperature may help kill the virus that causes warts. Thermo-insulation can also maintain the local temperature of the skin at higher and more stable temperatures, which in turn can help reduce variation in drug absorption as well as promote the delivery of the drug into or across the skin.

Alternatively, heating devices can be used to heat the skin and/or the cavity patch (including the drug-containing formulation/gel-triggering agent) to achieve enhanced results related to drug absorption, drug delivery from a depot beneath the skin, reducing onset time, or other benefits related to adding heat to a transdermal delivery system. Exemplary heating devices and appropriate uses for such heating devices that can be used in accordance with embodiments of the present invention are described in U.S. Pat. Nos. 6,955,819, 6,780,426, 6,756,053, 6,726,673, 6,613,350, 6,546,281, 6,488,959, 6,465,006, 6,453,648, 6,340,472, 6,306,431, 6,303,142, 6,284,266, 6,261,595, 6,245,347, and 5,919,479, each of which are incorporated herein by reference to the extent they are compatible with embodiments of the present invention.

The viscosity of the drug-containing composition used in the present invention should be low enough so that it can be placed in the open cavity of the cavity patch with ease, e.g., squeezed out of a flexible container. On the other hand, the viscosity can be high enough so that it does not easily spill or leak outside of the open cavity before gelling into a soft, coherent solid. Preferably, the viscosity of the drug-containing composition (solution or dispersion) can be from 200 to 2,000,000 centipoise, and more preferably, from 2,000 to 500,000 centipoise. In some formulation, the gelling agent alone may provide adequate viscosity. For formulations in which the gelling agent alone cannot provide adequate viscosity, the desired viscosity can be achieved by adding into the formulation appropriate amounts of one or more viscosity modifying agents, including but not limited to polyvinyl alcohol, ethyl cellulose, hydroxy propyl cellulose, a carbomer, a methacrylic polymer, and a methacrylate polymer. These viscosity modifying agents can be merely to modify viscosity, or can also double for the gelling agent, e.g., polyvinyl alcohol.

In one embodiment, the drug-containing composition can be an aqueous clear solution, an aqueous suspension, an emulsion (oil-in-water or water-in-oil), or other suspension containing suspended solid particles. The term “oil” as used herein refers to any liquid that is not appreciably soluble in water. Examples of oils which can be used in accordance with the present invention include but are not limited to isostearic acid, oleic acid, olive oil, vegetable oils, and/or essential oils.

The suspended solid particles may be substances for controlled release of the drug, such as ion-exchange resins. The drug containing composition can include additional solvents or liquid components such as propylene glycol, poly ethylene glycol, glycerol, polysorbate(s), and/or sorbitan ester(s). The additional liquids can serve various purposes such as solublizing the drug, reducing skin irritation, and/or improving the appearance of the composition.

The cavity patch of the present invention can be used to administer a wide variety of drugs and therapeutic compounds for treating a wide variety of conditions. Non-limiting examples of drugs and classes of drugs which can be administered using the cavity patch of the present invention include: those capable of activating the immune system such as imiquimod; antiviral agents; local and central nervous system acting analgesic and anesthetics such as lidocaine, fentanyl, and sufentanil; antifungal agents; skin cancer treatment agents; psoriasis treatment agents; common and genital warts treatments agents; keratolytics such as salicylic acid, alpha hydroxy acids (e.g., citric acid, lactic acid, malic acid, tartaric acid, glycolic acid), urea, benzoyl peroxide, tretinoin, sulfur, and rescorinol; smoking cessation agents such as nicotine; acne agents such as clindamycin and benzoyl peroxide; skin dispigmentation agents; combinations of ketamine/local anesthetics, combinations of ketamine/tricyclic antidepressants for the treatment of neuropathic pain; and local anesthetics with an NSAID for the treatment of neuropathic pain, muscle pain, and arthritis pain. Drugs used in the drug-containing composition of the present invention can be targeted for systemic delivery, regional delivery, and/or delivery into human skin. Additional applications for the cavity patch of the current invention could also include delivery of wound healing compounds, reduction and treatment of keloids, treatment of senile or solar lentgines, treatment of actinic keratosis, or other sundry cosmetic applications.

It is noteworthy that some drug-containing compositions are more effective when occluded because occlusion can keep beneficial volatile ingredients or solvent(s) in the formulation and/or improve drug skin penetration by keeping the skin well hydrated. Those formulations are particularly benefited when they are administered using the systems of the present invention. The cavity patch of the present invention can be configured to occlude the drug-containing compositions used therein.

As a further note, the formation of the water-based formulation into a soft, coherent solid after being placed into an open cavity patch can include a delicate balance between amount of gel-triggering agent,e.g., sodium borate, cavity thickness, gelling agent concentration, e.g., polyvinyl alcohol, composition or solution pH, and viscosity. Using boric acid and polyvinyl alcohol as an example, the boric acid (gel-triggering agent) provides for the gelling of the drug formulation. If the boric acid concentration is too low, the driving force for boric acid diffusion across the drug formulation in the cavity is reduced because of the lower concentration of borate ions dissolved at the aqueous formulation/nonwoven fabric interface. Cavity thickness can also impact the length of time the borate ions diffuse throughout the formulation in the cavity to gel the formulation into a solid. Increased concentrations of polyvinyl alcohol can increase the time for borate ions to diffuse throughout an increasingly viscous formulation in the cavity to gel the formulation as a whole, and “consume” borate ions in the process. Additionally, molecular weight for polyvinyl alcohol can also affect the gelling properties. Polyvinyl alcohol (PVA), USP (U.S. Pharmacopia), has a molecular weight from about 30,000 Mw to 50,000 Mw, and thus, the Examples provided utilize PVA within this range. However, molecular weights outside of this range also can form acceptable soft, coherent solids in accordance with embodiments of the present invention. Further, solution pH below 5 may result in incomplete solidification of the formulation because it is believed that the hydrogen ion content is high enough to compete with the polyvinyl alcohol in the complexation reaction with borate ions. Though certain parameters have been discovered as preferred, any combination of drug-containing composition and gel-triggering agent that works in the context of use in a cavity patch in accordance with embodiments of the present invention is included herein. This being stated, the impact of each of the above-mentioned parameters are explored in the Examples to provide the best known compositions, methods, and systems of practicing the present invention.

### EXAMPLES

### Example 1

A cavity patch is prepared having a configuration similar to that in FIG. 1 (top loading). A drug-containing composition (solution) containing an active drug and 15% polyvinyl alcohol in water. The cavity patch contains an absorbent mesh material which is a thin layer of absorbent gauze impregnated with about 5 mg/cm² sodium borate, and which is adhered on the underside of the ring-shaped patch wall (made from a soft, flexible foam tape) such that the mesh covers, but does not close, the lower end of the cavity in the ring. An impermeable cover is made of an impermeable tape (such as the 1525 adhesive tape by 3M) with a release liner covering the adhesive side.

To use the system, the user first affixes the cavity patch onto the skin so that the adhesive layer firmly seals the bottom of the patch onto the skin. Eight one hundredth of a milliliter of the drug-containing composition is dispensed onto the mesh and spreads over the skin area defined by the cavity. The user then pulls the release liner on the impermeable cover horizontally to seal the upper end of the open cavity with the cover. The solution is now sealed in the space defined by the skin, the cover, and the walls defining the once open cavity. After 20-30 minutes, the solution forms a soft, coherent solid gel while it is delivering drug into the skin, into the surrounding tissue, or into systemic circulation. The drug is delivered to the user's body continuously before and after the formulation is gelled. When the intended administration period is over, the entire loaded patch is simply removed form the skin. No residue from the drug-containing composition is left on the skin when the patch is removed since the drug-containing composition has gelled into a soft solid.

### Example 2

The drug-containing composition (solution) and materials in the cavity patch are the same as those in Example 1, but the configuration of the cavity patch is the bottom-loading type as shown in FIG. 2. An amount of drug-containing composition is dispensed into the open cavity of the cavity patch prior to adhesion of the patch to the skin. After the drug-containing solution is dispensed into the open cavity, the user affixes the cavity patch to the skin using the adhesive layer which is coated on the bottom of the cavity patch wall. This seals the patch to the skin and seals the drug-containing composition in the open cavity. The drug-containing composition gels into a soft solid and leaves no residue when the cavity patch and gel are removed at the end of the intended administration period.

### Example 3

Four cavity patches having the configuration as shown in FIG. 2 and containing 0.5, 1, 2, and 4 mg/cm² borate are studied to determine an optimal amount of borate (a gel-triggering agent) needed to gel a 15% polyvinyl alcohol (PVA) (a gelling agent) solution. The open cavity is 3 mm deep by 14 mm in diameter, and the absorbent material is a thin layer of nonwoven film impregnated with borate. The nonwoven material is affixed to the underside of the impermeable cover (3M 1523 polyethylene film) such that the nonwoven film covers the entire area of the open cavity. Specifically, the cavity patches containing 0.5, 1, 2, and 4 mg/cm² borate are each dosed with approximately 0.5 mL of a 15% PVA in water formulation, so that the filled open cavity has roughly 50 mg PVA per cm². The borate to PVA ratios per unit area for the patches are approximately 1:100, 2:100, 4:100 and 8:100, respectively. Each patch is then affixed to the upper arm of a study volunteer. After 3 hours, one side of the patch is lifted off the skin to observe the extent of PVA gelling in the open cavity. Only the patch containing 4 mg/cm² borate is gelled, this patch is then completely removed and there is no residue remaining on the arm. After 5 hours the remaining three patches are observed again, and the patch containing 2 mg/ cm² borate has mostly gelled, upon removal of this patch, a thin layer of PVA solution the diameter of the skin contact region remains on the arm. The patches containing 0.5 and 1 mg/cm² borate are not gelled and are removed.

Although the patches containing 0.5 and 1 mg/ cm² borate do not gel within 5 hours, they may gel after longer waiting times. However, prolonged gelling time increases the risk of spilling and thus is less desirable. Therefore, it is preferable to have sufficient amount of borate and appropriate concentrations of PVA in the system (see examples below) so that the formulation is gelled within 5 hours, and most preferably within 2 hours. From the data of this example, the ratio of borate to PVA is preferably greater than 2:100, more preferably greater than 4:100, and most preferably greater than 8:100, in order to achieve reasonably short gelling times.

As exemplified, an appropriate amount of borate impregnated in the nonwoven material can be used to ensure complete or substantially complete gelling of the drug formulations during the intended application time. The observed delay in gelling or lack of gelling of the PVA in the patches with borate concentrations below 2 mg/cm² can be explained by two factors: 1) there is not enough free borate available to undergo the interaction with the PVA, and 2) the driving force for borate diffusion across the open cavity is reduced because of the lower concentration of borate ions dissolved at the aqueous formulation/nonwoven fabric interface.

### Example 4

Three cavity patches with 14 mm diameter open cavities of different depths are prepared. The three depths are 0.13 mm, 0.26 mm, 1 mm, and 3 mm, respectively. A borate concentration of 4 mg/cm² is placed in each cavity patch. The cavity patches used are similar to that shown in FIG. 2 (bottom loading). The nonwoven material is affixed to the underside of the impermeable cover (3M 1523 polyethylene film) such that the nonwoven film covers the entire area of the open cavity. The purpose of this example is to study the length of time needed to gel a 25% PVA in water solution in each of the patches.

Each of the 4 patches is affixed to the upper arm of a study volunteer. After 30 minutes, one side of the patch is lifted off the skin to observe the extent of PVA gelling in the open cavity. After 30 minutes, the compositions in the patches with 0.13 mm and 0.26 mm deep open cavity patches are gelled. The patches are then removed. After 1.5 hours the 1 mm deep open cavity is gelled. After 3 hours the composition in the patch with a 3 mm deep open cavity is gelled.

The depth of the open cavity in the cavity patch has an impact on the time for a formulation to gel. Complexation of PVA in an aqueous solution in the presence of borate ions is dependent on the dissolution and diffusion of borate from the nonwoven material into the PVA solution. The longer gelling time for the 3 mm patch configuration may be due to the length of time required for the borate to diffuse throughout the formulation in the cavity to gel the formulation into a soft solid. Therefore, although formulations filled in open cavities deeper than 3 mm may still gel after long waiting time, such patch depth might be undesirable in some circumstances, as it increases the risk of spilling in short duration uses. Such patch depths may be practical in situations where longer uses are desirable.

### Example 5

A cavity patch similar to that shown in FIG. 2 (bottom loading) with a 3 mm depth and a 14 mm diameter having a borate concentration of 4 mg/cm² is studied to determine the length of time needed to gel 15%, 25%, and 40% PVA in water formulations. Again, the nonwoven material is affixed to the underside of the impermeable cover (3M 1523 polyethylene film) such that the nonwoven film covers the entire area of the open cavity. Each of the formulations is dosed into a cavity patch and is then affixed to the upper arm of a study volunteer. After 2, 3, 4, and 5 hours, one side of the patch is lifted off the skin to observe the extent of PVA gelling in the open cavity. The 15% PVA in water formulation is gelled at 2 hours, the 25% PVA formulation gelled at 3 hours, and the 40% PVA formulation had not gelled after 5 hours of wear. In each instance, upon removal of the patch after gelling was observed, no formulation residue is observed on the skin.

The concentration of PVA in an aqueous solution impacts the gelling time. Complexation of PVA in an aqueous solution in the presence of borate ions is dependent on the dissolution and diffusion of borate from the nonwoven material into the PVA solution. The longer gelling time as a function of increased PVA concentration is due to the length of time required for the borate to diffuse throughout an increasingly viscous formulation in the open cavity to gel the formulation into a solid, and “consumption” of the borate ions by PVA during the diffusion process. On the other hand, formulations with PVA concentrations lower than 8%, and especially those lower than 5%, tend to have difficulty solidifying into a solid with satisfactory strength. Therefore, the concentration of PVA in accordance with embodiments of the present invention is preferably in the range of 5 to 40%, and more preferably in the range of 8 to 30%.

### Example 6

A cavity patch having a configuration similar to that in FIG. 2 is prepared. Two placebo drug-containing compositions are studied. Composition A contains 12% PVA, 19% ISA, 2% trolamine, and 0.3% TR-2 (an emulsifier), and 66.7% water, with a pH of 7. Composition B contains 12% PVA, 19% ISA, 0.3% TR-2 (an emulsifier), and 68.7% water with a pH of 5.5. The open cavity in each of the two cavity patches is 3 mm deep with a 14 mm diameter, and the absorbent material is a thin layer of nonwoven film impregnated with 5 mg/cm² of borate. The nonwoven film is affixed to the underside of the impermeable cover (3M 1523 polyethylene film) such that the nonwoven film covers the entire area of the open cavity.

The system is used by dosing approximately 0.5 g of each placebo drug-containing composition into the open cavity, removing the release liner from the adhesive layer, and affixing the adhesive layer quickly to the skin. After 5 hours, the patches are evaluated to determine if the compositions have gelled into a solid. Composition A forms a soft solid gel and upon removal, there is no residue from the composition remaining on the skin. On the other hand, composition B does not form a gel after 5 hours. The patch is inspected again after 7 hours, and the formulation still has not formed a solid.

These results indicated that the gelling process of PVA in the presence of borate is dependent on pH. An optimal pH range for PVA gelling in the presence of borate is 6-9 (pHs higher than 9 may cause skin irritation, though these pH levels are still workable). Without being bound by any particular theory, at pH values lower than 5, the hydrogen ion concentration may be high enough to compete with PVA in the complexation reaction with borate ions.

### Example 7

A cavity patch similar to that shown in FIG. 2 (bottom loading) is prepared. An active drug-containing composition (Composition A) containing 12% PVA, 19% ISA, 2% trolamine, 4% imiquimod, 0.3% TR-2 (an emulsifier), and 62.7% water is dosed into the open cavity (3 mm deep and 14 mm diameter). The mesh coated material is a nonwoven impregnated with 4 mg/cm² borate as the gel triggering agent. The impermeable cover was a 3M 1523 polyethylene film, and the open cavity is formed with a foam tape. Approximately 0.5 g of drug solution is dosed into the open cavity. A release liner is removed exposing the adhesive layer on the bottom of the patch and the patch is immediately affixed to hairless mouse skin (HMS) previously mounted on a Franz diffusion cell. After approximately 4 hours, the patch is removed and the drug solution in the open cavity has formed a solid gel. No residue is left on the skin and the drug solution has not migrated from the area defined by the foam tape.

### Example 8

The formulation described in Example 7 is compared to Aldara (3M) for average skin flux in a hairless mouse skin (HMS) in vitro model. The HMS is mounted carefully between the donor and receiver chambers of a Franz diffusion cell. The receiver chamber is filled with pH 7.4 phosphate buffered saline (PBS). The experiment is initiated by placing the Example 7 formulation noted above in the open cavity (with 5 mg/cm² sodium borate) on the stratum corneum (SC) side of the skin sample. The Aldara is applied without sodium borate as directed. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time.

Table 1 shows data obtained using the experimental process outlined above.

TABLE 1
Steady-state flux (J) of Imiquimod through HMS

| | J |
|---|---|
| Formulation | (μg/cm²/h)* |
| Aldara | 0.21 ± 0.03 |
| Example 7 gelled with Na Borate | 0.32 ± 0.09 |

*Flux value is the average and standard deviation of three determinations.

### Example 9

The formulations, materials, and experimental design are the same as in Examples 2-7, but the configuration of the cavity patch is the top loading type as shown in FIG. 1. Due to the configuration of the cavity patch, drug-containing solution is placed into the open cavity after the cavity patch is affixed to the skin as taught in Example 1.

### Examples 10-13

Several cavity patches with a gel-triggering agent impregnated therein are prepared in accordance with embodiments of the present invention (top loading or bottom loading), and four drug-containing compositions are prepared in accordance with Table 2, as follows:

TABLE 2

Example

| | | | |
|---|---|---|---|
| 10 | 11 | 12 | 13 |

% by Weight

| | | | | |
|---|---|---|---|---|
| Polyvinyl alcohol (PVA) | 10.9 | 11.1 | 10.7 | 10.8 |
| Water | 61.8 | 62.9 | 60.5 | 61.2 |
| Ultrez 10 | 0.1 | 0.5 | 0.3 | 0.05 |
| Isostearic acid | 18.9 | 18.5 | 21.7 | 19.1 |
| Pemulen TR-2 | 0.3 | 0.3 | 0.3 | 0.3 |
| Trolamine | 3.6 | 2.3 | 2.1 | 4.25 |
| Imiquimod | 4.4 | 4.4 | 4.4 | 4.3 |

In each of Examples 10-13, the materials in Table 2 are combined according to the following procedure. The polyvinyl alcohol (PVA)and water are combined in a glass jar and heated with stirring until the PVA has dissolved. The Ultrez 10 is added to the PVA/water mixture and the mixture is stirred until the Ultrez dissolves into the solution. The isostearic acid and Pemulen TR-2 are then added to the solution and the mixture is stirred until the TR-2 is dissolved and a white solution is formed. Trolamine and imiquimod are then added and the entire mixture is vigorously mixed at room temperature.

### Examples 14-15

Two cavity patches with a gel-triggering agent impregnated therein are prepared in accordance with embodiments of the present invention (top loading or bottom loading), and two drug-containing compositions are prepared in accordance with Table 3, as follows:

TABLE 3

Example

| | |
|---|---|
| 14 | 15 |

% by Weight

| | | |
|---|---|---|
| Polyvinyl alcohol | 11.1 | 11.1 |
| Water | 62.7 | 63.2 |
| Carbopol 980 | 0.9 | 0.1 |
| Isostearic acid | 18.3 | 18.7 |
| Pemulen TR-2 | 0.3 | 0.3 |
| Trolamine | 2.2 | 2.2 |
| Imiquimod | 4.5 | 4.4 |

In each of Examples 14-15, the materials in Table 3 are combined according to the following procedure. The polyvinyl alcohol (PVA) and water are combined in a glass jar and heated with stirring until the PVA has dissolved. Carbopol 980 is added to the PVA/water mixture and the mixture is stirred until the Carbopol dissolves into the solution. The isostearic acid and Pemulen TR-2 are then added to the solution and the mixture is stirred until the TR-2 is dissolved and a white solution is formed. Trolamine and imiquimod are then added and the entire mixture is vigorously mixed at room temperature.

### Example 16-17

Two cavity patches with a gel-triggering agent impregnated therein are prepared in accordance with embodiments of the present invention (top loading or bottom loading), and two drug-containing compositions are prepared in accordance with Table 4, as follows:

TABLE 4

Example

| | |
|---|---|
| 16 | 17 |

% by Weight

| | | |
|---|---|---|
| Polyvinyl alcohol | 11.0 | 11.2 |
| Water | 62.28 | 63.2 |
| Carbopol 981 | 0.9 | 0.1 |
| Isostearic acid | 19.0 | 18.5 |
| Pemulen TR-2 | 0.22 | 0.3 |
| Trolamine | 1.9 | 2.2 |
| Imiquimod | 4.7 | 4.5 |

In each of Examples 16-17, the materials in Table 4 are combined according to the following procedure. The polyvinyl alcohol (PVA) and water are combined in a glass jar and heated with stirring until the PVA has dissolved. Carbopol 981 is added to the PVA/water mixture and the mixture is stirred until the Carbopol dissolves into the solution. The isostearic acid and Pemulen TR-2 are then added to the solution and the mixture is stirred until the TR-2 is dissolved and a white solution is formed. Trolamine and imiquimod are then added and the entire mixture is vigorously mixed at room temperature.

### Example 18

A cavity patch with a gel-triggering agent impregnated therein is prepared in accordance with embodiments of the present invention (top loading or bottom loading), and a drug-containing compositions is prepared in accordance with Table 5, which included a keratolytic agent (benzoyl peroxide):

TABLE 5

| | Example |
|---|---|
| | 18 |
| | % by Weight |
| Polyvinyl alcohol | 11.0 |
| Water | 62.22 |
| Ultrez 10 | 0.05 |
| Isostearic acid | 18.0 |
| Pemulen TR-2 | 0.2 |
| Trolamine | 2.04 |
| Benzoyl Peroxide | 2.8 |
| Imiquimod | 3.7 |

The materials in Table 5 are combined according to the following procedure. The polyvinyl alcohol (PVA) and water are combined in a glass jar and heated with stirring until the PVA had dissolved. The isostearic acid and Pemulen TR-2 are then added to the PVA/water solution and the mixture is stirred until the TR-2 is dissolved and a white solution is formed. Benzoyl peroxide is added and then enough trolamine is added dropwise until the pH is 6.5-7.0. The entire mixture is then vigorously mixed at room temperature.

### Example 19

The formulations of Examples 10-13, 15, 17, and 18 are tested in a human cadaver skin in vitro model. Human epidermal membrane (HEM) is used as the model membrane for the in vitro flux studies described herein. The HEM is mounted carefully between the donor and receiver chambers of a Franz diffusion cell. The receiver chamber is filled with pH 7.4 phosphate buffered saline (PBS). The experiment is initiated by placing each of the test formulations (10-13,15, 17, and 18) in an open cavity having 5 mg/cm² sodium borate impregnated on a non-woven fabric (bottom loading) on the stratum corneum (SC) of the skin sample. For comparison purposes, a product known as Aldara (3M) which is often used to treat genital warts, is also tested (without using a cavity patch). Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time.

Table 6 shows data obtained using the experimental process outlined above.

TABLE 6
Steady-state flux (J) of Imiquimod through HEM

| | J |
|---|---|
| Formulation | (μg/cm²/h) |
| Example 10 gelled with Na Borate | 1.1 ± 0.5 |
| Example 11 gelled with Na Borate | 1.7 ± 0.3 |
| Example 12 gelled with Na Borate | 1.4 ± 0.09 |
| Example 13 gelled with Na Borate | 0.2 ± 0.08 |
| Example 15 gelled with Na Borate | 2 ± 2 |
| Example 17 gelled with Na Borate | 0.2 ± 0.03 |
| Example 18 gelled with Na Borate | 0.1 ± 0.07 |

The formulations of the invention shown above generally provide for significant penetration of the active ingredient, and further, these values were found to be comparable to the marketed product Aldara.

### Example 20

Viscosity values reported below are obtained by conventional method used by those skilled in the art. The measurement of the viscosity of the example formulations are compared to a control sample containing only polyvinyl alcohol (PVA) and water and are run under the same conditions. Comparison to the control is illustrative. The viscosity values are obtained using a Brookfield RVDV-1+viscometer with an S-15 spindle (viscosity values reported at 2 rpm and at 25 C). The following examples are provided to illustrate advantages of certain embodiments of the present invention, but are not intended to be limiting thereof.

TABLE 7
Viscosity values of Examples

| Example | Viscosity @ 2 rpm |
|---|---|
| 10 | 69500 |
| 11 | 20250 |
| 12 | 27750 |
| 13 | 32500 |
| 14 | 55000 |
| 15 | 30000 |
| 17 | 12500 |
| 18 | 7250 |
| Control* | 7350 |

*Control patch consisted of all the components in Example 10 except the thickening agent (Ultrez 10, Carbopol 980, or Carbopol 981) was not included.

The formulations of Table 7 all had sufficient viscosity such that the compositions did not spill when placed in the empty patch cavity and applied to a skin surface.

### Example 21

A circular ring is cut out of a medical grade foam tape (3M 1779), which has a thickness of 1/16 inch. The ring's inner and outer diameters are ⅜ inch and ¾ inch, respectively. A gauze disc with a diameter of ½ inch is impregnated with 10 mg sodium borate (gel-triggering agent). A disc with a diameter of ¾ inch is cut out of another medical grade foam tape with a thickness of 1/32 inch (3M9773). The ring, the gauze disc, and the foam tape disc are co-centrally assembled into an open cavity patch configuration such that the foam tape disc completely covers the non-adhesive side of the ring, and the gauze disc is sandwiched between the ring and the foam tape disc. The ring's adhesive side is thus still open, and the gauze is visible from the opening. The space defined by the inner space of the ring and the gauzed lined foam tape disc is the medicine cavity. The cavity patch is placed on a release liner with the adhesive side resting on the release liner (with multiple cavity patches), similar to that shown in FIG. 2.

Separately, an oil-in-water viscous fluid formulation (wart treatment formulation) is then prepared which includes 3 wt % imiquimod, 10 wt % benzoyl peroxide, 15 wt % polyvinyl alcohol (gelling agent), 0.5 wt % Carbopol 981 (thickening agent), 0.2 wt % sodium hydroxide (agent for neutralizing Carbopol 974), 10 wt % petrolatum (oil phase), 5 wt % stearyl alcohol (oil phase), 5 wt % polysorbate (emulsifying agent), and 51.3 wt % water and is loaded into a standard 20 mL aluminum ointment tube with a screw cap.

To use, the cavity patch is removed from the release liner, which exposes the opening of the medicine cavity. About 0.1 mL of the wart treatment formulation is placed in the medicine cavity, and the filled patch is covered over the wart. In this configuration, the wart becomes essentially completely submerged in the formulation, and the patch is firmly secured on the skin via adhesive on the ring. During the treatment, water occluded in the medicine cavity hydrates the wart surface for the duration that the patch is on the wart, the salicylic acid performs its function of loosening the keratin and desquamation, and imiquimod activates the local immune system to fight against the wart virus. After a predetermined time period, e.g., 12 hours, 24 hours, or more, the patch is removed from the wart. Since the formulation has long been gelled into a soft, coherent solid, substantially no residual formulation is left on the skin. The above process may be repeated over and over again until a desired effect is achieved.

### Example 22

A cavity patch is prepared as described in Example 21, except that the mesh impregnated with sodium borate is fastened to the ring, and an occlusive membrane with one side coated with an adhesive and covered with a release liner is on a non-adhesive side of the ring. In other words, the ring is applied to the skin, the medicine is applied to the wart within the ring, and the occlusive membrane is applied to the ring, enclosing the wart within the filled cavity, similar to that shown in FIG. 1.

In this embodiment, the wart treatment formulation is the same, except that the polyvinyl alcohol concentration is 10 wt % and the water content is 56.1 wt % (less viscous than that in Example 21). To use the system, the user places the cavity patch (without the occlusive backing adhered thereto) on the wart so that the entire wart is inside the ring. Thus, the adhesive coated on the bottom of the ring affixes and seals the bottom of the ring on the skin. The wart treatment formulation is placed in the medicine cavity, and the release liner on the occlusive membrane is pulled horizontally, guiding the occlusive backing onto the ring structure as the release liner is pulled away. The adhesive on the occlusive membrane seals the medicine cavity. Alternatively, the occlusive backing/release liner can be a completely separate structure than the ring, where the release liner is removed from the occlusive backing, and the occlusive backing is carefully placed on the ring to ensure a substantially air tight seal within the medicine cavity.

### Example 23

The present system includes three elements: a wart treatment formulation, a cavity patch, and a heating unit. The wart treatment formulation and the cavity patch are the same as that in Example 22 or Example 23. The heating unit is a iron oxidation based device described in U.S. Pat. No. 6,453,648. The heating unit has an area approximately the size of the cavity patch (or slightly larger), and is placed on top of the occlusive membrane either as an integral part of the cavity patch or as independent unit that can be placed on the occlusive membrane (which can be separately adhered thereto). The heat increases local skin temperature by a few degrees, e.g., to about 37° C. from typical 32° C.) which not only helps control the wart virus (as this virus prefers cooler temperatures), but also increases the hydration effect and permeation flux of the drug(s) into the wart surface.

### Example 24

Three cavity patches are prepared which can be used for cessation of nicotine addiction, e.g., smoking, tobacco, etc. The configuration of the cavity patches can be made to be similar to that in Example 1-9 (top loading or bottom loading). In this example, the cavity patches are similar to that in Example 1, except that the open cavities in the cavity patches all have a depth of 0.3 mm and areas of 5 cm², 10 cm², and 15 cm², respectively. The active drug in the drug-containing composition is nicotine for cessation of smoking or other tobacco dependency. In one embodiment, the 15 cm² patch is to be used for the first phase of treatment, the 10 cm² patch for the second phase of treatment, and the 5 cm² patch for the third phase of treatment.

### Example 25

A cavity patch is prepared which can be used to delivery fentanyl or sufentanil. The configuration of the cavity patches can be made to be similar to that in Example 1-9 (top loading or bottom loading). In this example, the cavity patch is similar to that in Example 1, except that the open cavity in the cavity patch has a depth of 0.3 mm, and depending on the dosage of fentanyl or sufentanil to be delivered, an area of 5cm², 10 cm², or 15 cm². The concentration of the fentanyl or sufentanil in the drug-containing composition, the concentration of solvent in the drug containing composition, and the area of skin contact provided by the cavity patch can all affect the amount and rate of drug delivery.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A system for dermal delivery of a drug, comprising:
a gel-triggering agent;
a drug-containing composition including a drug and a gelling agent, said drug-containing composition forming a soft, coherent solid when contacted with the gel-triggering agent; and
a cavity patch having an open cavity configured to be closed at least in part by a skin surface, said open cavity further configured to facilitate contact between the skin surface and the soft, coherent solid,
wherein the gel-triggering agent and the drug-containing composition are positioned in the system such that they are kept isolated from one another until immediately before or during use.

2. The system of claim 1, wherein said cavity patch includes an impermeable cover and at least one patch wall which define said open cavity.

3. The system of claim 2, wherein said impermeable cover comprises an adhesive substrate.

4. The system of claim 2, wherein said impermeable cover has thermo-insulation properties.

5. The system of claim 2, wherein said patch wall comprises a foam tape.

6. The system of claim 1, wherein the cavity patch has a thickness from 0.1 mm to 5 mm.

7. The system of claim 1, wherein the cavity patch has a thickness from 0.2 mm to 3 mm.

8. The system of claim 1, wherein said drug is imiquimod.

9. The system of claim 1, wherein said drug is nicotine.

10. The system of claim 1, wherein said drug is an antiviral agent.

11. The system of claim 1, wherein said drug is an analgesic.

12. The system of claim 1, wherein the drug is selected from the group of sufentanil and fentanyl.

13. The system of claim 1, wherein said drug is an antifungal agent.

14. The system of claim 1, wherein said drug is a drug for treating skin cancer.

15. The system of claim 1, wherein said drug is a drug for treating warts.

16. The system of claim 1, wherein said drug is a keratolytic agent.

17. The system of claim 16, wherein said keratolytic agent is selected from the group of salicylic acid, alpha hydroxy acids, urea, benzoyl peroxide, tretinoin, sulfur, rescorinol, trichloroacetic acid, and combinations thereof.

18. The system of claim 1, wherein said drug is a drug for treating genital warts.

19. The system of claim 1, wherein said drug is a drug for treating plantar warts.

20. The system of claim 1, wherein said drug is targeted for systemic delivery.

21. The system of claim 1, wherein said drug is targeted for regional delivery.

22. The system of claim 1, wherein said drug is targeted for skin delivery including regions of the skin into and below the epidermis.

23. The system of claim 1, wherein the cavity patch is configured to occlude the drug-containing composition.

24. The system of claim 1, wherein the soft, coherent solid formed upon contacting the gel-triggering agent and the gelling agent leaves no residue on the skin when it is removed.

25. The system of claim 1, wherein said gelling agent is a polyvinyl alcohol.

26. The system of claim 25, wherein the concentration of the polyvinyl alcohol in the drug-containing composition is from 5% to 40% by weight.

27. The system of claim 25, wherein the concentration of the polyvinyl alcohol in the drug-containing composition is from 8% to 20% by weight.

28. The system of claim 1, wherein the gel-triggering agent is impregnated into an absorbent material disposed within the open cavity.

29. The system of claim 3, wherein the gel-triggering agent is coated onto the adhesive coated substrate of the impermeable cover.

30. The system of claim 1, wherein the gel-triggering agent is coated on the cavity patch within the open cavity.

31. The system of claim 1, wherein the gel-triggering agent is specific for gelling the drug-containing composition when it includes polyvinyl alcohol as the gelling agent.

32. The system of claim 1, wherein the gel-triggering agent is boric acid, a salt of boric acid, or a borate.

33. The system of claim 32, wherein the boric acid, the salt of boric acid, or the borate is impregnated into or coated on an absorbent material at a quantity from 1 to 20 mg/cm².

34. The system of claim 32, wherein the boric acid, the salt of boric acid, or the borate is impregnated into or coated on an absorbent material at a quantity of more than 0.5 mg/cm².

35. The system of claim 1, wherein the drug-containing composition comprises water.

36. The system of claim 1, wherein the drug-containing composition is an emulsion.

37. The system in claim 1, wherein the skin surface which closes at least in part the open cavity forms a skin contact region which has an area from 0.1 cm² to 20 cm².

38. The system in claim 37, wherein the area of the skin contact region is from 0.2 cm² to 10 cm².

39. The system of claim 1, wherein the drug-containing composition comprises a viscosity modifying agent.

40. The system of claim 39, wherein the viscosity modifying agent is selected from the group consisting of polyvinyl alcohol, ethyl cellulose, hydroxy propyl cellulose, a carbomer, a methacrylic polymer and a methacrylate polymer.

41. The system of claim 1, wherein said drug-containing composition comprises an oil phase and an aqueous phase.

42. The system of claim 1, wherein said drug-containing composition comprises an emulsifying agent

43. The system of claim 1, wherein said drug-containing composition comprises isostearic acid.

44. The system of claim 1, wherein said drug-containing composition comprises oleic acid.

45. The system of claim 1, wherein said drug containing composition comprises olive oil.

46. The system of claim 1, wherein said drug-containing composition comprises one or more solvents selected from the group of propylene glycol, poly ethylene glycol, glycerol, polysorbate, and sorbitan ester(s).

47. The system of claim 1, wherein said drug-containing composition comprises glycerol.

48. The system of claim 1, wherein said drug-containing composition comprises a polysorbate.

49. The system of claim 1, wherein said drug-containing composition comprises a sorbitan ester.

50. The system of claim 1, wherein said drug-containing composition has a pH greater than 5.

51. The system of claim 1, wherein said drug-containing composition has a pH from 6 to 9.

52. The system of claim 1, wherein the cavity patch is top-loading.

53. The system of claim 1, wherein the cavity patch is bottom-loading.

54. A method for dermal delivery of a drug, comprising
affixing a cavity patch to a skin surface, said cavity patch having an open cavity configured to be closed at least in part by the skin surface;
contacting a drug-containing composition comprising a drug and a gelling agent with the gel-triggering agent such that a soft, coherent solid is formed within the cavity patch; and
closing the open cavity.

55. The method of claim 54, wherein an amount of drug-containing composition is disposed in the open cavity prior to affixing the cavity patch to the skin.

56. The method of claim 54, wherein an amount of drug-containing composition is disposed in the open cavity after the cavity patch is affixed to the skin.

57. The method of claim 54, wherein said drug is imiquimod.

58. The method of claim 54, wherein said drug is nicotine.

59. The method of claim 54, wherein said drug is an antiviral agent.

60. The method of claim 54, wherein said drug is an analgesic.

61. The method of claim 54, wherein said drug is selected from the group of fentanyl and sufentanil.

62. The method of claim 54, wherein said drug is an antifungal agent.

63. The method of claim 54, wherein said drug is a drug for treating skin cancer.

64. The method of claim 54, wherein said drug is a drug for treating warts.

65. The method of claim 54, wherein said drug is a drug for treating genital warts.

66. The method of claim 54, wherein said drug is a drug for treating plantar warts

67. The method of claim 54, wherein said drug is a drug for treating pain.

68. The method of claim 54, wherein said drug is targeted for systemic delivery.

69. The method of claim 54, wherein said drug is targeted for regional delivery.

70. The method of claim 54, wherein said drug is targeted for skin delivery including regions of the skin into and below the epidermis.

71. The method of claim 54, wherein the soft, coherent solid formed upon contacting the gel-triggering agent and the gelling agent leaves no residue on the skin when it is removed.

72. The method of claim 54, wherein said gelling agent is a polyvinyl alcohol (PVA).

73. The method of claim 72, wherein the concentration of the polyvinyl alcohol in the drug-containing composition is from 5 to 40% by weight.

74. The method of claim 72, wherein the concentration of the polyvinyl alcohol in the drug-containing composition is from 8 to 20% by weight.

75. The method of claim 54, wherein the gel-triggering agent is impregnated into or coated on an absorbent material disposed within the open cavity.

76. The method of claim 54, wherein the gel-triggering agent is coated on the cavity patch within the open cavity.

77. The method of claim 54, wherein the gel-triggering agent is specific for gelling the drug-containing composition when it includes polyvinyl alcohol as the gelling agent.

78. The method of claim 54, wherein the gel-triggering agent is boric acid, a salt of boric acid, or a borate.

79. The method of claim 78, wherein the boric acid, the salt of boric acid, or the borate is impregnated into or coated on an absorbent material at a quantity from 1 to 20 mg/cm².

80. The method of claim 78, wherein the boric acid, the salt of boric acid, or the borate is impregnated into or coated on an absorbent material at a quantity of more than 0.5 mg/cm².

81. The method of claim 54, wherein the drug-containing composition is an emulsion.

82. The method of claim 54, wherein the skin surface which closes at least in part the open cavity forms a skin contact region having an area from 0.1 cm² to 20 cm².

83. The method of claim 82, the skin contact region has an area from 0.2 cm² and 10 cm².

84. The method of claim 54, wherein the drug-containing composition comprises a viscosity modifying agent.

85. The method of claim 84, wherein the viscosity modifying agent is selected from the group consisting of polyvinyl alcohol, ethyl cellulose, hydroxy propyl cellulose, a carbomer, a methacrylic polymer and a methacrylate polymer.

86. The method of claim 54, wherein said drug-containing composition comprises one or more solvents selected from the group of propylene glycol, poly ethylene glycol, polysorbate, and sorbitan ester(s).

87. The method of claim 54, wherein said drug-containing composition has a pH greater than 5.

88. The method of claim 54, wherein said drug-containing composition has a pH from 6 to 9.

89. The method of claim 51, wherein the step of closing the cavity includes placing the cavity patch on the skin surface, and after the drug-containing composition and the gel-triggering agent are within the cavity patch, placing an impermeable cover over an open end of the cavity patch.

90. The method of claim 54, wherein the cavity patch includes an impermeable cover and side walls such that after the drug-containing composition and the gel-triggering agent are disposed within the open cavity, the step of closing the cavity patch occurs when the cavity patch is placed on the skin surface.

91. A system for dermal delivery of a drug, comprising:
a cavity patch having an open cavity configured to be closed at least in part by a skin surface;
a gel-triggering agent disposed within the open cavity; and
a drug-containing composition including a drug and a gelling agent, said drug-containing composition forming a soft, coherent solid when contacted with the gel-triggering agent;
wherein the gel-triggering agent and the drug-containing composition are positioned in the system such that they are kept isolated from one another until immediately before or during use.

92. The system of claim 91, wherein said cavity patch includes an impermeable cover and at least one patch wall which define said open cavity.

93. The system of claim 91, wherein said drug is selected from the group of imiquimod, nicotine, an antiviral agent, an analgesic such as fentanyl or sufentanil, an antifungal agent, a keratolytic agent, a drug for treating skin cancer, a drug for treating warts, or combinations thereof.

94. The system of claim 91, wherein the soft, coherent solid formed upon contacting the gel-triggering agent and the gelling agent leaves no residue on the skin when it is removed.

95. The system of claim 91, wherein the gel-triggering agent is impregnated into or coated on an absorbent material disposed within the open cavity.

96. The system of claim 91, wherein said gelling agent is a polyvinyl alcohol.

97. The system of claim 91, wherein the gel-triggering agent is specific for gelling the drug-containing composition when it includes polyvinyl alcohol as the gelling agent.

98. The system of claim 91, wherein the gel-triggering agent is boric acid, a salt of boric acid, or a borate.

99. A device for facilitating dermal delivery of a drug, comprising:
a cavity patch having an open cavity which, when closed, is defined by one or more side wall, an impermeable top cover, and a skin surface; and
a material within the cavity having a gel-triggering agent impregnated within or coated on the material,
said device configured to be devoid of drug until immediately prior to or during use.

100. A device as in claim 99, wherein the device is top loading, and the occlusive cover is configured to close the open cavity after application of the device to the skin surface.

101. A device as in claim 99, wherein the device is bottom loading, and the occlusive cover is attached to the wall such that attachment of the device to the skin surface closes the open cavity.

102. A device as in claim 99, wherein the material is a non-woven material.

103. A device as in claim 99, wherein the material is gauze.

104. A device as in claim 99, wherein the material is a mesh.

105. A device as in claim 99, wherein the gel-triggering agent is specific for gelling a composition including polyvinyl alcohol.

106. A device as in claim 99, wherein the gel-triggering agent is boric acid, a salt of boric acid, or a borate.
